(19) United States
(12) Patent Application Publication
(10) Pub. No.: US 2004/0220516 A1
(43) Pub. Date: Nov. 4, 2004

(54) Food extraction apparatus and method

(76) Inventors: Stephen Solomon, Baltimore, MD (US); Moshe Shike, Larchmont, NY (US); Sam Klein, Clayton, MO (US)

Correspondence Address: FRISHAUF, HOLTZ, GOODMAN & CHICK, PC, 767 THIRD AVENUE, 25TH FLOOR, NEW YORK, NY, 10017-2023, US

(21) Appl. No.: 10/702,194

(22) Filed: Nov. 4, 2003

Related U.S. Application Data
(60) Provisional application No. 60/423,645, filed on Nov. 4, 2002.

(51) Int. Cl. A61M 31/00

(52) U.S. Cl. 604/66; 606/151; 604/174

(57) ABSTRACT

An apparatus is provided which includes (i) a transabdominal tube having a proximal end portion and a distal end portion, and (ii) a pump that is attachable to the distal end portion of the tube. The tube is inserted into a patient such that the proximal end portion of the tube is disposed in the upper digestive system of the patient and the distal end portion of the tube extends externally from the patient. The pump is connected to the distal end portion of the tube, and the pump is controlled to remove partially digested food from the upper digestive system of the patient.

### FIELD OF THE INVENTION

This invention relates to an apparatus and method for removing partially digested food from a patient.

### BACKGROUND OF THE INVENTION

Obesity is a major health problem in the United States and Europe. The National Health and Nutrition Examination Survey (1988-1994) reported that approximately 20-25% of Americans are obese, while another study estimated the percentage of overweight Americans to be between 60% and 65% (Flegal K M, Carroll M D, Ogden C L, Johnson C L. “Prevalence and trends in obesity among US adults, 1999-2000”. JAMA. 2002; 288:1723-1727). Obesity accounts for numerous health problems, including diabetes, degenerative joint disease, hypertension, and heart disease.

Weight reduction can be achieved by increased caloric expenditure through exercise and/or by reduced caloric consumption through diet. When combined, these therapies can result in a 5-10% weight loss over 4-6 months. However, in most cases weight gain often recurs, and improvements in related comorbidities are often not sustained (Mitka M, “Surgery for obesity: Demand soars amid scientific, ethical questions”. JAMA. 2003; 289:1761-2).

Surgical procedures present an increasingly common solution for morbidly obese patients. Morbid obesity is a condition characterized by a Body Mass Index (BMI) of at least 40 kg/m² or by a BMI of at least 35 kg/m² with the presence of one or more comorbidities. Surgical procedures include both gastric restrictive operations and malabsorptive operations and will be performed in an estimated 98,000 patients in 2003 (Mitka, supra).

U.S. Pat. No. 5,345,949 to Shlain discloses a method and devices for laparoscopically performing a gastric restrictive operation known as stapled gastroplasty. In Shlain, a band is placed around the stomach such that the stomach is divided into a proximal pouch and a distal pouch with an aperture therebetween for allowing proximal pouch contents to pass to the distal pouch and the remainder of the digestive system. The band is then stapled into place, with the staples penetrating at least a superficial part of the stomach. In an alternate embodiment of the Shlain procedure, a clamp is used instead of a band to divide the stomach and is then stapled to prevent migration.

U.S. Pat. No. 6,572,629 to Kalloo et al discloses a method for performing banded gastroplasty. As in Shlain, the stomach is divided into proximal and distal pouches. Kalloo et al, however, discloses an alternative method for creating the pouches wherein a ligating loop is attached to an exterior or interior wall of the stomach. The ligating loop is then constricted to reduce stomach capacity at the base of the esophagus.

Gastroplasty procedures encounter many problems. For example, stapling gastroplasty procedures such as the invention of Shlain may encounter rupture of the staple line or postoperative hernia. In addition, many patients who undergo banded gastroplasty procedures such as the one disclosed by Kalloo et al regain a substantial portion of the lost weight within 3-5 years. Additionally, gastroplasty has a 15-20% reoperation rate due to either stomal outlet stenosis or severe gastroesophageal reflux (Brolin, RE, “Bariatric surgery and long-term control of morbid obesity.” JAMA. 288:2793-6). Still further, lastly gastroplasty is not easily reversed and the procedure is complex and expensive to perform.

U.S. Pat. No. 6,511,490 to Robert discloses a method and device invention directed to a further type of gastric restrictive operation known as gastric banding. The goal of the gastric banding operation is essentially the same as that of gastroplasty. A proximal pouch at the base of the esophagus is created by encircling an inflatable band around the stomach. The band is inflated by injecting an inflation fluid into a subcutaneously implanted injection port. In invention of Robert, the size of the stoma between the proximal and distal stomach pouches may be advantageously manipulated by varying the volume of the inflation fluid in the inflation band.

Gastric banding such as the method and device disclosed in Robert, however, encounters problems such as deterioration in the usability of the inflatable bands. In addition, patient weight loss with gastric banding is less reliable than weight loss documented with other surgical weight loss procedures (Brolin, supra).

The most common surgical procedure in the treatment of morbid obesity is gastric bypass surgery. Roux-en Y Gastric Bypass (RYGB) entails the creation of a small pouch with the proximal stomach with a volume of 10-30 cc's. Using a Roux-en Y gastrojejunostomy, the jejunum is connected to the proximal stomach pouch.

Complications of gastric bypass surgery, however, include venous thrombosis or pulmonary embolism (1%-2%), anastomotic leaks (1% 2%), and wound infection (1%-5%). In addition, iron and vitamin B12 deficiencies occur in more than 30% of patients and half of the patients with iron deficiency develop a microcytic anemia (Brolin, supra).

Another increasingly common procedure for treatment of morbid obesity is bilopancreatic bypass (BPB). BPB involves mild restriction of stomach capacity. The entire jejunum is bypassed by connecting a proximal section of the duodenum to the ileum at a point 50-100 cm proximal to the ileocecal junction. Variants on the bilopancreatic procedure include the distal Roux-ex Y procedure, in which the Roux limb is connected to the distal bowel. These surgical procedures, however, are the most invasive and risky.

Ideally there should be a less invasive procedure that is easily performed and more easily reversed.

### OBJECT OF THE INVENTION

It is an object of the present invention to provide an apparatus and method for treating morbid obesity through a non-surgical approach.

### SUMMARY OF THE INVENTION

According to one embodiment of the invention, an apparatus is provided which comprises: (i) a transabdominal tube having a proximal end portion adapted to be inserted into the upper digestive system of a patient and a distal end portion adapted to extend externally from the patient, and (ii) a pump that is attachable to the distal end portion of the tube for removing partially digested food from the patient.

### BRIEF DESCRIPTION OF THE DRAWINGS

FIG. 1 is a schematic view of the tube of the apparatus in a patient;

FIG. 1A is a schematic view of an alternate embodiment of the tube of the apparatus;

FIG. 1B is a schematic view of another alternate embodiment of the tube of the apparatus;

FIG. 1C is a cross sectional schematic view of the tube of the apparatus taken perpendicular to the axis of the tube;

FIG. 2 is a schematic view of a pump of the apparatus;

FIG. 3 is a schematic view of an electrical air compressor for use with the pump shown in FIG. 2;

FIG. 4 is a schematic view of the apparatus with a manual bulb pump;

FIG. 5 is a schematic view of the apparatus with a syringe serving as a pump;

FIG. 6 is a schematic view of the apparatus with internal sensors;

FIG. 7 is a schematic view of the apparatus with a bag;

FIG. 8 is a schematic view of the apparatus with a cleaning device;

FIG. 9 is a schematic view of the apparatus with an inflated balloon anchor;

FIG. 10 is an axial cross sectional schematic view showing one-way valves provided in the lumens of the tube of the apparatus;

FIG. 11 is a schematic view of a second embodiment of the present invention.

### DETAILED DESCRIPTION

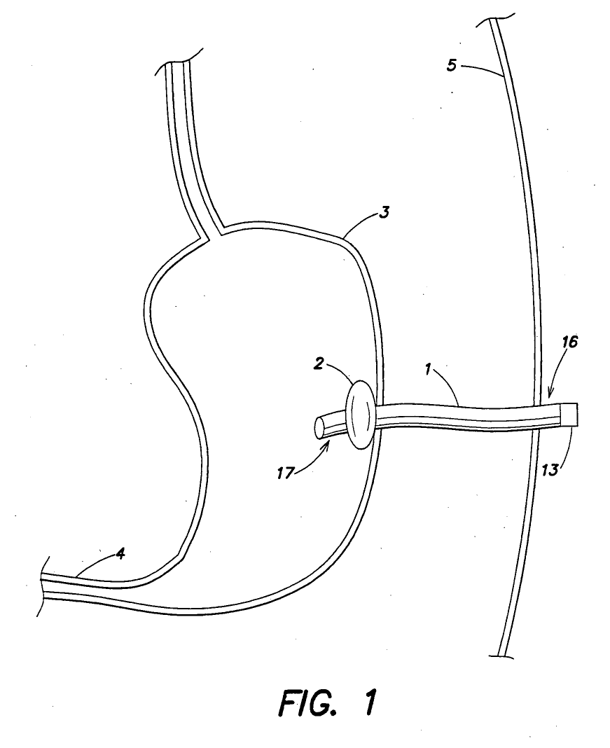

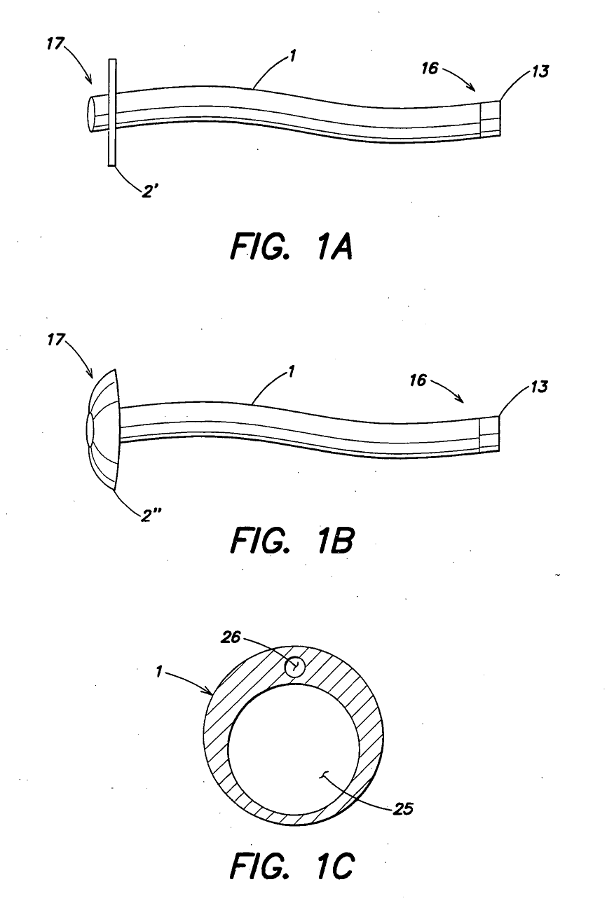

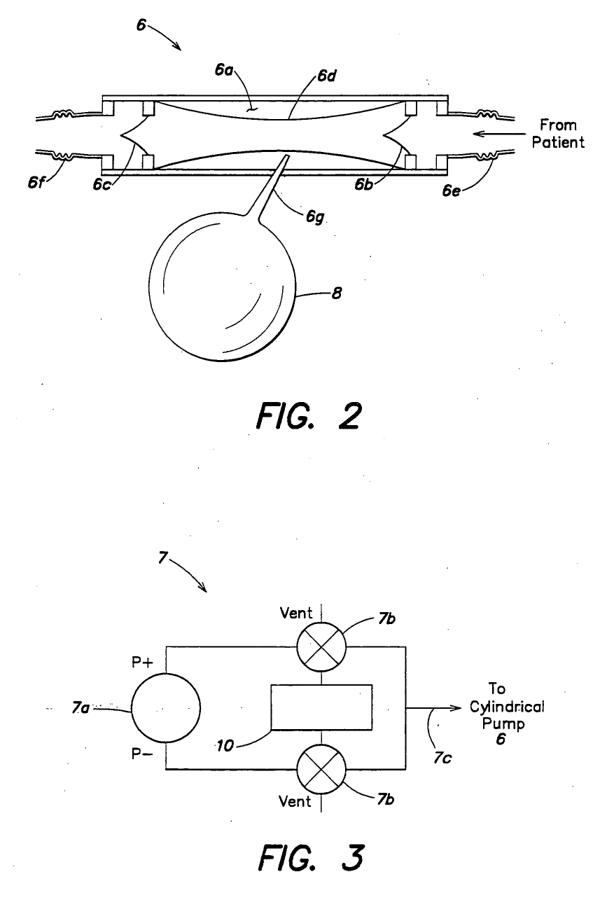

FIG. 1 shows a transabdominal tube 1 placed in a patient such that a proximal end portion 17 of the tube 1 is disposed inside the stomach 3 of a patient and a distal end portion 16 of the tube 1 extends out from the skin 5 of the patient. The tube 1 is ideally 28 French and is preferably non-collapsible. A retention member, an inflation portion 2 (balloon anchor) in this example, is provided at the proximal end portion 17 of the tube 1 to prevent the tube 1 from coming out of the stomach 3. A cap 13 is detachably provided at the end of the distal end portion 16 and seals the tube 1 when it is attached. The cap 13 is removed when a pump 6, 8, 9 (FIGS. 2, 4, 5, respectively) is attached to the tube 1 to remove food from the upper digestive system of the patient.

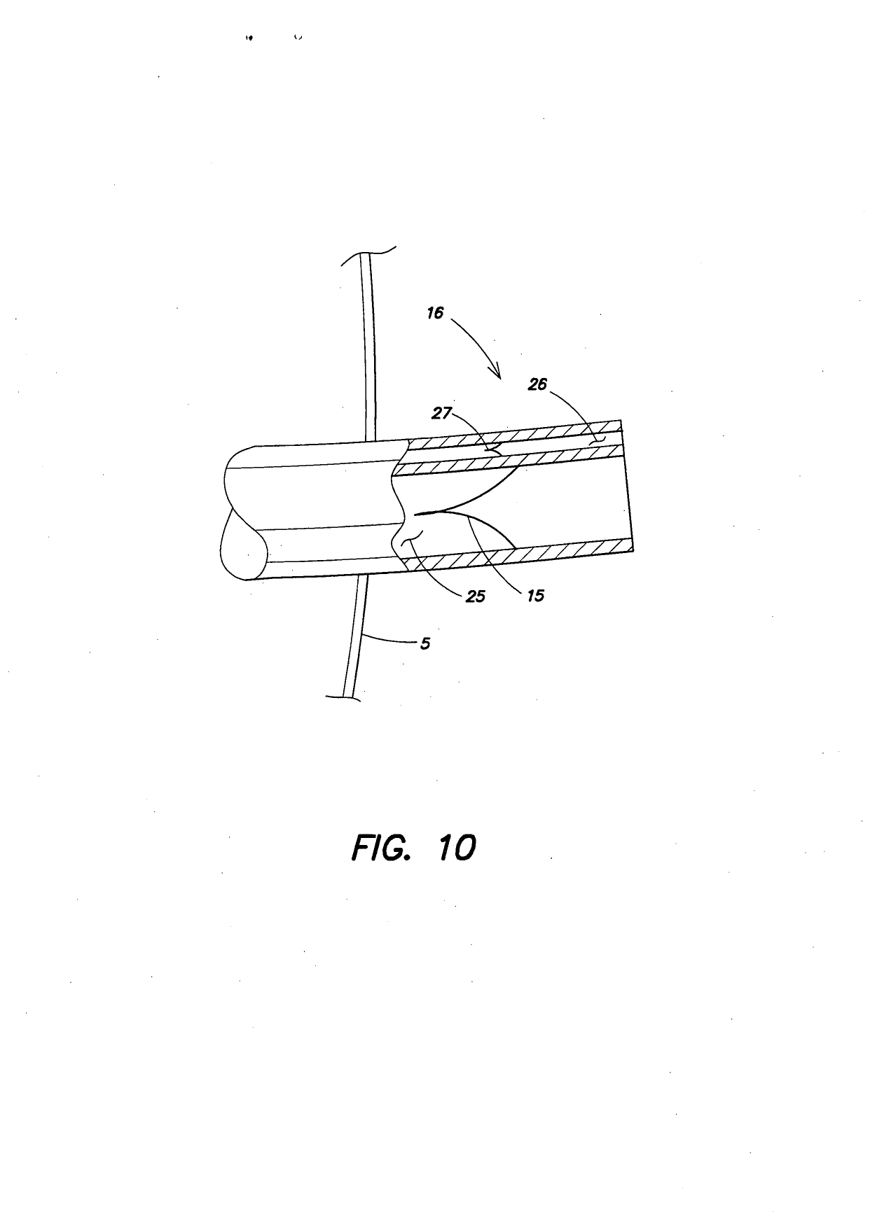

FIG. 1C shows a cross section of tube 1 taken perpendicular to the axis of tube 1. Removal lumen 25 extends from the proximal end portion 17 to the distal end portion 16 of the tube 1 and is a pathway for the removal of partially digested food from the stomach 3 or other part of the upper digestive system of the patient. Inflation lumen 26 extends from the inflation portion 2 to the distal end portion 16 of the tube 1 and is a pathway for introducing fluid to the inflation portion 2 from outside of the patient. The inflation lumen 26 is preferably minimal in size to allow the removal lumen 25 to be as wide as possible within the tube 1. One way valves 15, 27 are provided in lumens 25, 26, respectively, as shown in FIG. 10.

As used herein, the term “partially digested food” includes any food which has been ingested by the patient, and the term “upper digestive system” includes the stomach 3, duodenum 4 and proximal jejunum of the patient.

Reference is now made to methods which may be used to insert the tube 1. These methods entail less risk of complications and less cost than conventional, surgical methods of treating obesity, and patients who undergo these treatments are typically discharged within one day of the operation. These methods are therefore especially advantageous for use in treating morbid obesity because such patients are at increased risk for surgical complications due to their obesity.

The tube 1 may be inserted, for example, via Percutaneous Endoscopic Gastrostomy (PEG). A variety of methods of performing PEG are well known in the art, and any one of the methods may be used to insert the tube 1. PEG procedures are successfully completed in over 90 percent of attempts. PEG is performed under conscious sedation induced by, for example, meperidine or midazolam. According to one method of PEG known as the pull method, an endoscope is inserted into the stomach through the mouth of the patient. The stomach is insufflated by blowing air into the stomach, either through the endoscope or via a nasogastric tube. The insufflation brings the stomach in apposition to the abdominal wall and allows for direct access from the skin to the stomach of the patient.

An insertion site is located by surveying the interior of the stomach with the endoscope. The endoscope is then used to illuminate the selected insertion site in such a way that the light of the endoscope is visible from outside of the patient's body through the skin of the patient.

An incision is made at the place on the patient's skin indicated by the light from the endoscope and at the corresponding location on the exterior wall of the stomach. A cannula is then inserted through the incisions and a guide wire is inserted into the stomach through the cannula. Graspers on the end of the endoscope grab hold of the proximal portion of the guide wire in the stomach and the endoscope is withdrawn from the patient while the graspers hold the guide wire. The guide wire is of sufficient length to allow a distal portion of it to extend out of the patient from the cannula after the proximal portion is withdrawn from the stomach and through the patient's mouth by the endoscope.

The end of the guide wire extending out from the patient's mouth is attached to the distal end of the tube 1, which is drawn though the mouth and esophagus and into the stomach of the patient by pulling on the distal end of the guide wire. The tube 1 is then pulled through the incisions in the stomach and skin of the patient until only the proximal end 17 and the inflation portion 2 of the tube 1 remain inside of the stomach. The cannula is removed as the distal end of the tube 1 is drawn through the incision in the stomach, and is removed entirely when the distal end of the tube 1 is disposed at the patient's skin. The inflation portion 2 of the tube 1 is then inflated by introducing fluid into the inflation portion 2 through the inflation lumen 26. The inflated inflation portion holds the tube 1 in place and the guide wire is removed from the tube 1.

An alternate method of PEG known as push PEG may also be used, for example, to insert the tube 1. The push method is similar to the pull method; however, according to the push method the guide wire is not attached to the tube 1. Instead, the tube 1 is pushed along the guide wire through the mouth and esophagus of the patient. The tube 1 is also pushed through the incisions in the stomach and the skin of the patient until it is disposed as described hereinabove with respect to the pull method.

A third method which may, for example, be used for inserting the tube 1 via PEG is known as the Russell method. As with both the push method and the pull method, the insertion site is located via endoscopy. An incision is made in the skin and stomach and a guide wire is inserted through the incision into the stomach via a cannula or needle. A dilator (or introducer) with a peel away sheath is guided along the guide wire and inserted into the stomach. After the dilator (introducer) and sheath are inside the gastric lumen, the dilator is removed and the tube 1 is inserted along the guide wire and through the peel away sheath. The sheath is then peeled away and the tube 1 is fixed in place.

The tube 1 may be also be inserted without using an endoscope, for example, through a procedure known as Percutaneous Radiological Gastrostomy (PRG). According to PRG, the stomach is insufflated via a nasogastric tube. Organs which may be interposed between the stomach and the abdominal wall, such as the colon, are excluded by CT scan or ultrasonography. Exclusion of interposed organs may also be accomplished after insufflation by fluoroscopy. The selection of the insertion site is also determined by fluoroscopy or a similar method.

After the insertion site has been located, the tube 1 may be inserted transabdominally as in the Russell method of PEG. Alternatively, a guide wire may be inserted as in the endoscopic pull method. The wire is then maneuvered through the stomach and esophagus and out of the patient's mouth and is used to guide the tube 1 back through the mouth, esophagus and stomach and out of the insertion site (Mustafa N. Özmen et al. “Percutaneous radiologic gastrostomy.” European Journal of Radiology. 43:186-95).

The tube 1 may also be inserted in other portions of the upper digestive system. For example, direct jejunostomy, wherein a tube is inserted transabdominally into the jejunum, may be accomplished through methods similar to those described hereinabove with reference to gastrostomy tube placement. The retention member of the device must be smaller for jejunostomy procedures, however, in order to avoid irritation of the jejunum or obstruction of the jejunal lumen.

It is not necessary for the tube 1 to comprise an inflation portion 2. Instead, the proximal end portion 17 of the tube 1 may comprise another type of retention member such as a flange 2′ (see FIG. 1A) or a dome 2″ (see FIG. 1B) to prevent the tube 1 from coming out of the stomach 3 or other section of the upper digestive system. With the retention members 2′ and 2″ of FIGS. 1A and 1B, the second lumen 26 in tube 1 can be eliminated.

Ideally, inflatable retention members are used in combination with transabdominal insertion of the tube 1, while either inflatable or rigid retention members are used in combination with procedures similar to the push and pull methods. One example of a tube which comprises an inflation member is disclosed by U.S. Pat. No. 6,506,179 to Tiefenthal et al, the entire contents of which are incorporated herein by reference. In addition, an additional deformable alternative retention member is disclosed in U.S. Pat. No. 6,077,250 to Snow et al, the entire contents of which are incorporated herein by reference.

Retention members which may be deformed in situ allow the tube 1 to be removed without additional endoscopy. The retention member is deflated or deformed and the tube 1 is pulled out using traction. In cases where the retention member is rigid, the tube 1 is either cut close to the skin and removed endoscopically or is cut near to the stomach and pushed into the stomach to allow spontaneous elimination with waste.

The stomach is held in apposition to the abdominal wall by, for example, insufflation during the tube placement procedure and by the retention member after the tube 1 has been placed. However, it may be preferable to anchor the stomach to the abdominal wall by gastropexy, which may prevent complications arising from tube placement and may facilitate the placement procedure. In addition, gastropexy is important in jejunostomy procedures in order to secure the jejunum during the tube placement procedure (Özmen et al, supra).

For example, to secure the stomach or jejunum to the abdominal wall, T-shaped metal or nylon fixing members are inserted trans-gastrically or trans-jejunally by syringe close to the tube insertion site. The fixing members assume a T shape after insertion and are tied near to the skin. Four fixing members are typically disposed in a square pattern around the tube insertion site to secure the stomach or jejunum. (See, for example, F. J. Thornton et al. “Percutaneous radiologic gastrostomy with and without T-fastener gastropexy: a randomized comparison study”. Cardiovasc Intervent Radiol. 2002 November-December; 25(6):467-71.)

Reference is now made to various forms of pumps which are attachable to the distal end portion 16 of the tube 1. FIGS. 2, 4 and 5, for example, display pumps 6, 8, 9 which are attachable to the distal end portion 16 of the tube 1 for removal of partially digested food from the stomach 3 or upper digestive system of the patient.

A preferred embodiment of a pumping device for use in the present invention is shown in FIG. 2. The cylindrical pump 6 comprises a semi-flexible outer cylinder 6a of silicone rubber or similar material that contains two one way valves 6b, 6c directing the stomach contents away from the patient's body. A thin flexible silicone rubber or similar material inner cylindrical membrane 6d is arranged in the outer cylinder 6a between the two one way valves 6b, 6c. Two barbed portions 6e,6f or other fittings are arranged at either end of the outer cylinder 6a. The space between the outer cylinder 6a and the inner membrane 6d can be alternately insufflated with air and evacuated through aperture 6g in the outer cylinder 6a. The proximal barbed portion 6e or other fitting is inserted into the distal end 16 of the stomach tube 1. When air is insufflated between the outer cylinder 6a and the thin membrane 6d, the thin membrane 6d collapses onto itself increasing the pressure within the chamber formed within the thin membrane 6d. This causes the distal one way valve 6c to open (to expel contents of the membrane 6d) while at the same time causing the proximal one way valve 6b to close. When the air is withdrawn (expelled) from between the cylindrical membrane 6d and the outer cylinder 6a, a negative pressure is created causing distal one way valve 6c to close and the proximal one way valve 6b to open. When valve 6b opens, the negative pressure aspirates the stomach contents through the stomach tube 1 into the chamber formed within the inner membrane cylinder 6d. When the cycle is repeated (i.e. air is introduced between cylinders 6a and 6d), the aspirated portion of the stomach contents now residing in the inner membrane cylinder 6d is expressed (expelled) via the distal one way valve 6c and the distal barbed portion 6f or other fitting. The alternating insufflation and evacuation of the space between the outer cylinder 6a and the inner membrane 6d will extract the stomach contents. A fixed volume of stomach contents, determined by the dimensions of the pumping apparatus heretofore described, is removed for every cycle of the pump. Hence, the flow rate and volume removed may be easily determined. The dimensions of this pumping device can be scaled to generate larger or smaller flow rates per pump cycle.

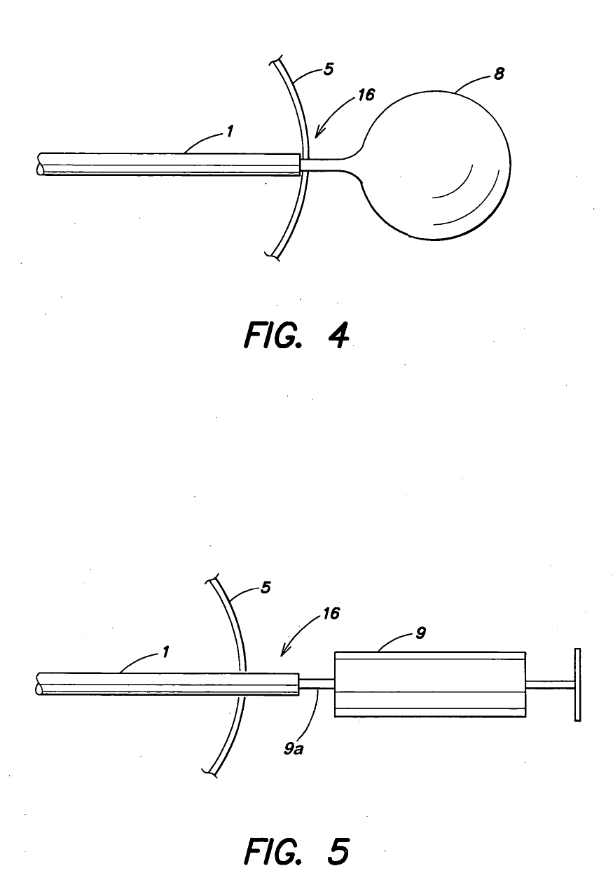

The alternating insufflation and evacuation of the space between the outer cylinder 6a and the inner membrane 6d can be generated in a variety of ways. A simple hand squeeze bulb 8 (see FIG. 4) having a fitting or tube 6g that air-tightly enters the space between the outer cylinder 6a and the inner membrane 6d, as shown in FIG. 2, may be used to operate the pump. Hand squeezing the bulb 8 pressurizes the space between the outer cylinder 6a and the inner membrane 6d. Alternatively, in the absence of a hand squeeze bulb, the outer cylinder 6a itself may be flexible and may be squeezed by hand to perform the pumping action.

FIG. 3 shows an electrical air compressor system 7 that is attachable to the cylindrical pump 6 to insufflate and evacuate the space between the outer cylinder 6a and the inner membrane 6d. The electrical air compressor system 7 includes a small electrically powered air pump 7a, solenoid valves 7b and electronic control circuitry (i.e., a CPU) 10 and may be used to automate the insufflation and evacuation process. The outlet 7c is air-tightly connected to the opening in the outer cylinder 6a in place of tube 6g. The controller 10 cycles the valves 7b to alternately provide air pressure and release the air pressure in pump 6, as described above. This implementation can also be used to control the quantity and rate at which the stomach contents are to be withdrawn.

A manual bulb pump 8 which is attachable to the distal end portion 16 of the tube 1 is shown in FIG. 4. The manual bulb pump 8 preferably comprises silicon rubber or a similar flexible material such so as to permit the contents of the bulb pump 8 to be evacuated by squeezing the bulbous end of the bulb pump 8. The circumference of a tapered end essentially corresponds to an interior circumference of the lumen 25 of the tube 1. To operate the manual bulb pump 8, air is first evacuated from the bulb pump 8 by squeezing the bulb, and then the tapered end of the bulb pump 8 is inserted into the lumen 25 of the distal end portion 16 of the tube 1 so as to create a seal between the tapered end and the tube 1. The bulb is then released to allow it to re-inflate. The negative pressure in the bulb pump 8 (when it is released) causes partially digested food to flow out from the upper digestive system toward the distal end portion 16 of the tube 1 and into the bulb of the manual bulb pump 8. The bulb pump 8 is then disengaged from the tube 1 and the removed partially digested food is evacuated from the bulb. The manual bulb pump 8 removes a predetermined volume of partially digested food during each cycle, thereby allowing the volume of removed food to be easily determined. The cycle may be repeated until a desired amount of partially digested food is removed from the upper digestive system of the patient.

A pump in the form of a syringe 9 which is attachable to the distal end portion 16 of the tube 1 is shown in FIG. 5. The syringe 9 preferably comprises a tapered end portion with an aperture at the distal end thereof. The circumference of the tapered end portion 9a preferably essentially corresponds to the interior circumference of the lumen 25 of the tube 1. To operate the syringe 9 to remove partially digested food from the upper digestive system of the patient, the contents (air or partially digested food) of syringe 9 are evacuated by depressing the plunger. The tapered end portion 9a of the syringe 9 is inserted into the distal end portion 16 of the tube 1 so as to create a seal between the tapered end portion 9a and the tube 1. The plunger of the syringe 9 is then withdrawn so as to create negative pressure to draw partially digested food out from the upper digestive system through the tube 1 and into the syringe 9. The syringe 9 is then disengaged from the tube 1 and evacuated by, for example, depressing the plunger thereof. As in the case of the cylindrical pump 6 and the manual bulb pump 8, the syringe 9 removes a predetermined volume of partially digested food during each cycle, thereby allowing the volume of removed food to be easily determined. The cycle may be repeated until a desired amount of partially digested food is removed from the upper digestive system of the patient.

The cylindrical pump 6, manual bulb pump 8 and syringe 9 are preferably activated by the patient or by a health care provider at a predetermined time after eating. The predetermined time is preferably set by a physician. A physician also preferably determines a maximum volume of partially digested food to be removed from the upper digestive system of the patient after each meal. The maximum volume is ideally set in terms of a maximum number of pumping cycles which is programmed into the CPU 10 of the electronic air compressor 7 or which is told to the patient or health care provider if the pump 6, 8, 9 is manually operated.

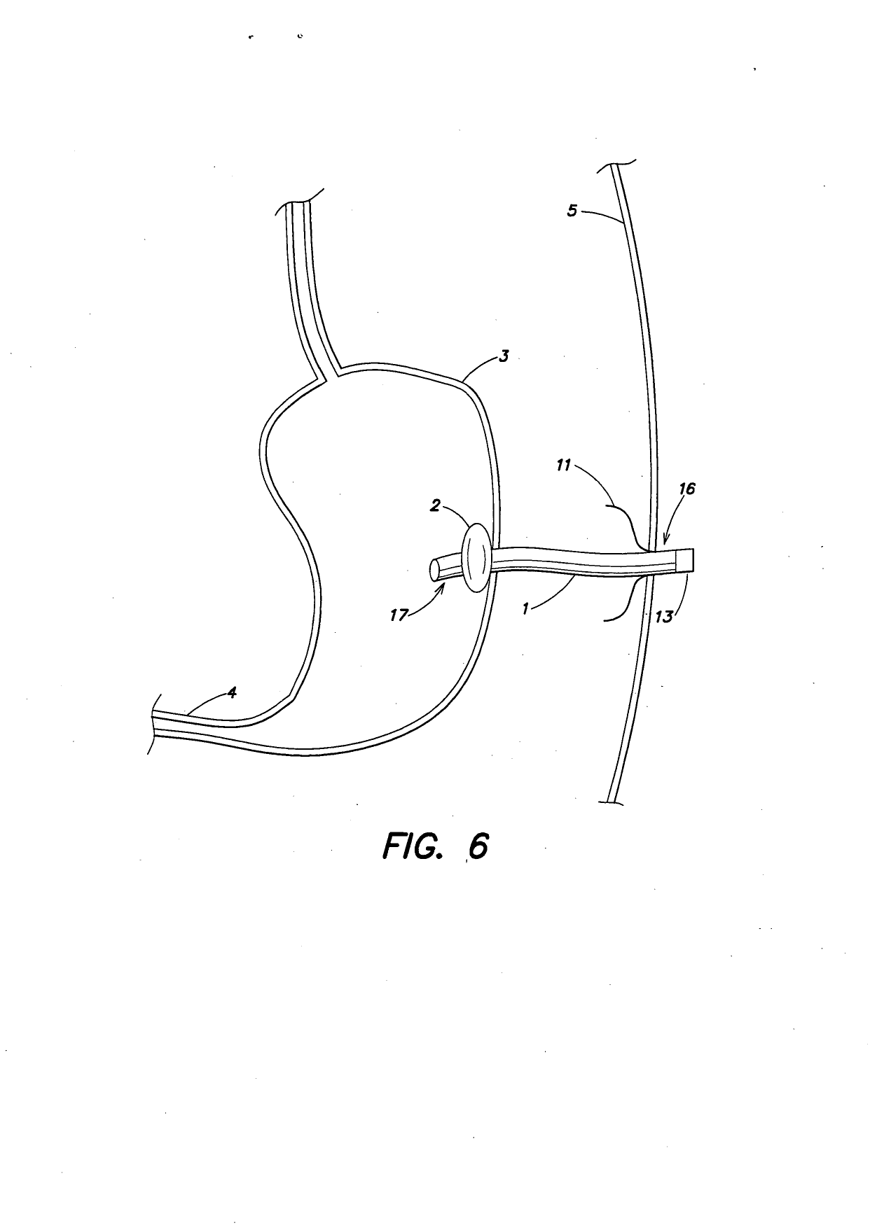

Patients who undergo treatment with the apparatus of the present invention are preferably monitored closely by a health care provider in order to ensure that the apparatus is working properly and that patient remains in good health. If the electronically controlled air compressor 7 is used to power the cylindrical pump 6, the CPU 10 of the compressor 7 preferably records data such as frequency of use of the pump and number of pumping cycles per use. In addition as shown in FIG. 6, sensors 11 may be placed in the patient's body, for example in a body cavity, capillary bed, or vein, and may extend along the tube 1. The sensors 11 extend out from the skin of the patient along, or inside the wall of, the tube 1 and end in electrodes at the distal end portion 16 of the tube 1. Electrodes on the electronic air compressor 7 preferably connect the CPU 10 of the electronic air compressor 7 to the sensors 11. Alternatively, the sensors 11 may be monitored during visits to a health care provider. The sensors 11 monitor the biochemical/nutritional status of the patient by measuring data such as metabolic, nutritional, and/or electrolyte levels and/or other chemical processes. A control section of the CPU 10 ideally deactivates the electrical air compressor 7 if any of the value(s) measured by sensor 11 exceeds a predetermined range. In addition, the CPU 10 ideally includes a transmitting section which transmits the recorded number of pumping cycles and/or the values detected by sensors 11 to a health care provider via, for example, a telephone line or the internet. The information detected by the sensors 11 is also preferably stored in a recording section of the CPU 10. The stored information is preferably downloaded via a telephone line or the internet by a health care provider and used to track use of the cylindrical pump 6 and to ensure that the patient remains healthy while using the apparatus.

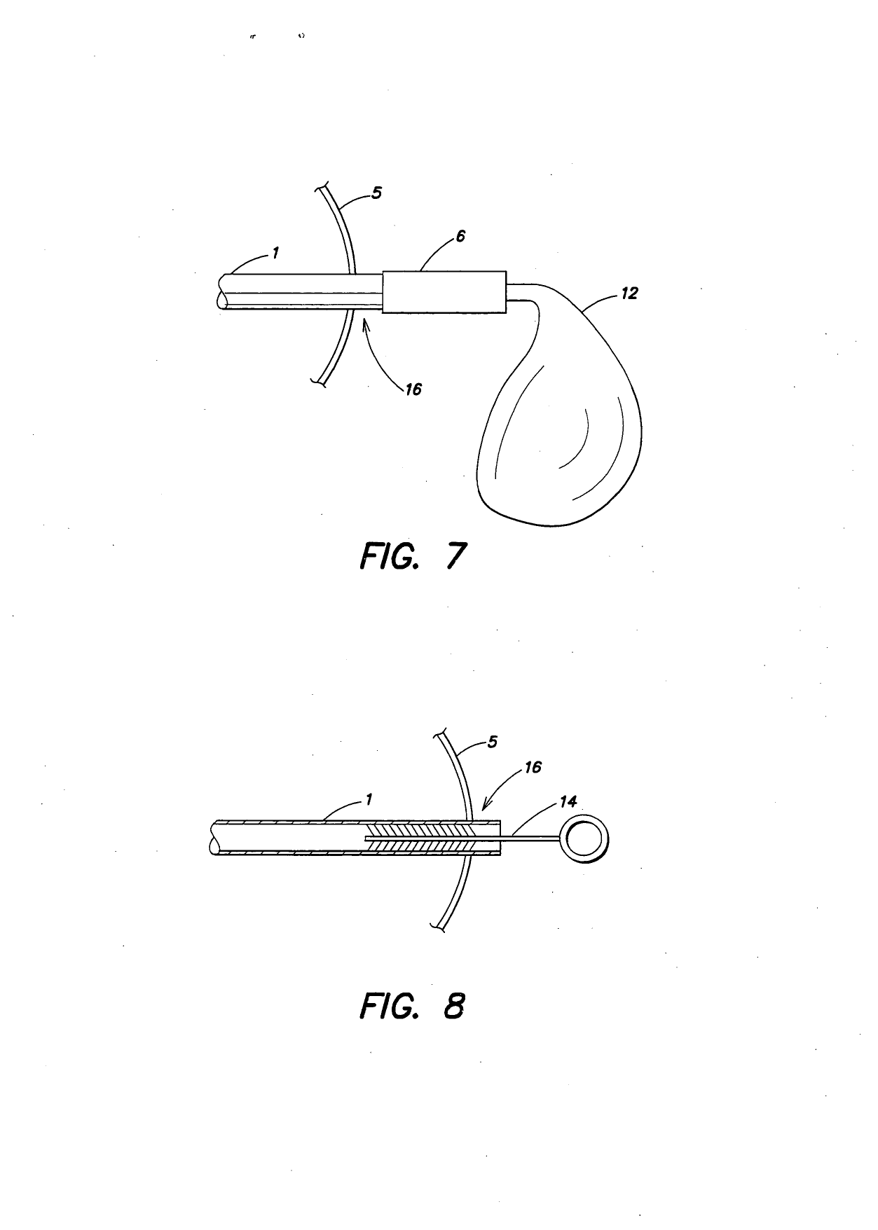

After the partially digested food is pumped out of the upper digestive system of the patient by the cylindrical pump 6, the food is preferably stored in a bag which is attachable to the distal barbed portion 6f of the cylindrical pump 6 as shown in FIG. 7. The bag is ideally opaque and scented and may be worn by the patient on a belt or other strap.

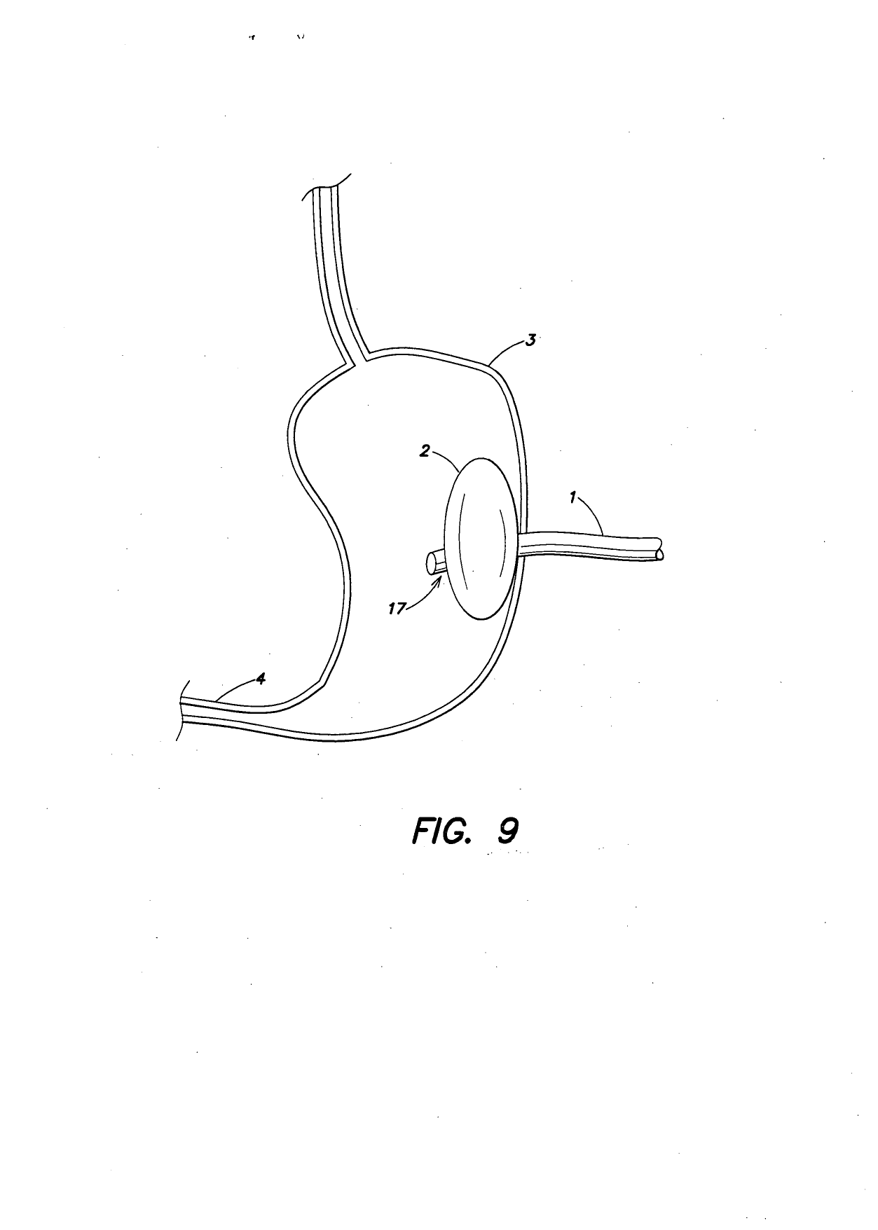

The tube 1 is preferably cleaned using a brush 14 which is adapted to clean the inside of the tube 1 as shown in FIG. 8. The cylindrical pump 6, manual bulb pump 8 and syringe 9 are preferably flushed with saline and/or a disinfectant solution after use.

In addition to removing food from the upper digestive system of a patient, the apparatus may also be used to decrease stomach capacity and create a feeling of satiety in the patient by inflating the inflation portion 2 as shown in FIG. 9. To achieve this purpose, the balloon anchor 2 may be variably inflated by a physician by adding or removing fluid through the inflation lumen 26 of the tube 1.

FIG. 10 shows an axial cross sectional view of the tube 1 extending out from the skin 5 of the patient in which removal lumen 25 and inflation lumen 26 are visible. In a feature which may be incorporated into any of the various embodiments of the present invention, a one way valve 15 is provided at the distal end portion 16 of the tube 1 in the removal lumen 25. The one way valve 15 is oriented to prevent partially digested food from leaving the tube 1. The valve 15 is opened when a pump is attached to the distal end portion 16 of the tube 1. For example, the barbed portion 6e of the cylindrical pump 6, the tapered end portion of the manual bulb pump 8 and the tapered end portion of the syringe 9 each push open the valve 15 when they are inserted into the distal end portion 16 of the tube 1. When the valve 15 is pushed open by the ends of the pumps, partially digested food can be removed as described hereinabove. When a pump is not attached to open valve 15, the cap 13 is preferably placed on the distal end portion 16 of the tube 1 to prevent any remaining drops of partially digested food from escaping from the tube 1. The cap 13 may be pressed onto the end of the tube 1, threaded on the end of the tube 1, or may have projections which are frictionally inserted into the ends of lumens 25, 26, to seal them in a closed condition.

FIG. 10 also shows a one way valve 27 provided at the distal end portion 16 of the tube 1 in the inflation lumen 26. The valve 27 prevents the fluid used to inflate the inflation portion 2 from escaping the inflation portion 2 through the inflation lumen 26. That is, one way valve 27 prevents inflation portion 2 from deflating. If it becomes necessary to deflate the inflation portion 2 to remove the tube 1 from the upper digestive system of the patient, or to further inflate the portion 2, a needle on a syringe is preferably inserted into the inflation portion 26 so as to open the valve 27 by pushing the needle through the valve members. The fluid used to inflate the inflation portion 2 may then be removed or added with the syringe.

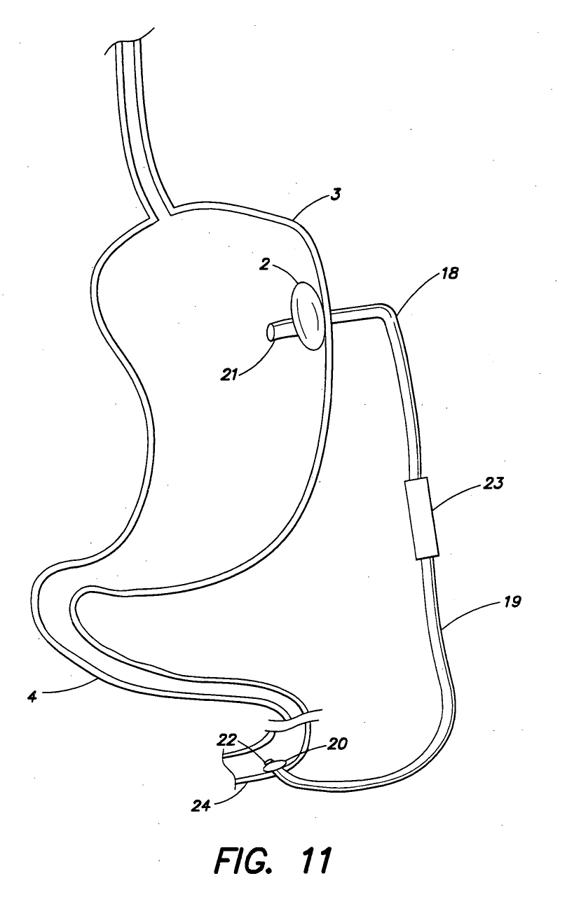

FIG. 11 shows an alternate embodiment of the present invention directed to intestinal bypass instead of the removal of partially digested food. A first tube 18 is inserted into the stomach 3 and is anchored in the stomach 3 by a balloon anchor 2 provided at a proximal end portion 21 of the tube 18. The procedure for tube placement is the same as described hereinabove with reference to the placement of the tube 1. A second tube 19 is inserted into the distal digestive system, for example the distal jejunum or the ileum 24 and is anchored by a second balloon anchor 20 provided at a proximal end portion 22 of the tube 19. The second tube 18 may be inserted endoscopically or radiologically as described hereinabove with reference to gastrostomy and jejunostomy. The tubes are connected subcutaneously by a connecting device 23 which preferably includes a one way valve such as one way valve 15 to direct food toward the tube 19. Connecting device 23 also preferably contains a pump for pumping partially digested food from the stomach 3 to the ileum 24. Such a pump may be an electrically operated pump or a manually operated pump operable via, for example, a tube extending out of the body of the patient.

It is noted that the food extraction apparatuses and methods of the present invention are preferably combined with a behavior modification program which ideally educates patients in modifying caloric intake, lifestyle and attitudes toward food. Learned activities and support for weight loss may include activities such as self-monitoring by recording food intake and physical activity, avoiding triggers that prompt eating, assistance from family and friends, problem solving skills and relapse prevention. The program may be taught by an instructor or offered over the internet. In addition, the program preferably includes a series of regular check-ups by a health care provider. The check-ups ideally include regularly testing blood for electrolytes, supplementing patients' diets with vitamins, and administering medications to prevent gallstone formation as needed. Ideally, the behavior modification program will educate patients to change their lifestyle so as to eliminate the need for food extraction.

Additional advantages and modifications will readily occur to those skilled in the art. For example, the features of any of the embodiments may be used singly or in combination with any other of the embodiments of the present invention. In addition, the insertion technique for placing the tube is not limited to known gastrostomy techniques. Accordingly, various modifications may be made without departing from the spirit or scope of the general inventive concept as defined by the appended claims and their equivalents.

What is claimed is:

1. An apparatus comprising:
a transabdominal tube having a proximal end portion adapted to be inserted into the upper digestive system of a patient and a distal end portion adapted to extend externally from the patient; and
a pump attachable to the tube for removing partially digested food from the upper digestive system of the patient.

2. The apparatus of claim 1, wherein the pump is attachable to the distal end portion of the tube.

3. The apparatus of claim 1, wherein the pump is removable from the tube and comprises at least one of a cylindrical pump, a bulb pump, and a syringe.

4. The apparatus of claim 3, wherein the pump is automated and the apparatus further comprises a control section to activate the pump.

5. The apparatus of claim 4, wherein:
the control section comprises sensors which detect at least one of a volume of the removed food and a biochemical/nutritional status of the patient; and
the control section deactivates the pump when one of (i) the detected volume exceeds preset parameters within a given time and (ii) the detected biochemical/nutritional status exceeds preset parameters.

6. The apparatus of claim 5, wherein control section transmits at least one of the detected volume and the detected biochemical/nutritional status to a health care provider.

7. The apparatus of claim 6, wherein the control section records detected information.

8. The apparatus of claim 3, wherein the pump is manually operable.

9. The apparatus of claim 1, further comprising a bag adapted to be attached to the pump;
and wherein the removed food is pumped into the bag.

10. The apparatus of claim 1, further comprising a cap for plugging the distal end portion of the tube when the pump is not attached to the tube.

11. The apparatus of claim 1, further comprising an anchor to hold the tube in the upper digestive system of the patient.

12. The apparatus of claim 11, wherein the anchor comprises a balloon anchor coupled to the tube and adapted to be anchored in the upper digestive system of the patient.

13. The apparatus of claim 12, wherein the balloon anchor is adapted to be variably inflated so as to selectively fill a desired portion of the stomach of the patient.

14. The apparatus of claim 12, wherein the tube includes an inflation lumen coupled to said balloon for communicating with the interior of the balloon.

15. The apparatus of claim 1, wherein the tube includes a one way valve adapted to prevent the partially digested food from unintentionally escaping from the tube;
and wherein the one way valve is adapted to be opened when the pump is attached to the tube.

16. A method comprising:
inserting a tube into a patient such that a proximal end portion of the tube is disposed in the upper digestive system of the patient and a distal end portion of the tube extends externally from the patient;
connecting a pump to the distal end portion of the tube; and
controlling the pump to remove partially digested food from the upper digestive system of the patient through the tube.

17. The method of claim 16, further comprising:
monitoring at least one of a volume of the pumped food and a biochemical/nutritional status of the patient;
ending the pumping when one of (i) the monitored volume exceeds preset parameters within a given time and (ii) the monitored biochemical/nutritional status exceeds preset parameters.

18. The method of claim 17, further comprising transmitting at least one of the volume of the pumped food and the biochemical/nutritional status of the patient to a health care provider.

19. The method of claim 16, further comprising at least one of:
educating the patient to modify caloric intake, lifestyle and food psychology through at least one of an instructor-taught program and an internet program;
regularly testing the patient's blood for electrolytes;
supplementing the patient's diet with vitamins as needed; and
administering medication to prevent gallstone formation as needed.

20. An apparatus comprising:
a first tube adapted to be inserted into the upper digestive system of a patient;
a second tube adapted to be inserted into the lower digestive system of the patient;
wherein the first and second tubes are adapted to be connected in a subcutaneous tunnel; and
wherein partially digested food is transported through the first tube and the second tube from the upper digestive system to the lower digestive system of the patient.
